(19) United States
(12) Patent Application Publication
(10) Pub. No.: US 2022/0328174 A1
(43) Pub. Date: Oct. 13, 2022

(54) CENTRALIZED SYSTEM FOR VACCINATION VERIFICATION, INVENTORY MANAGEMENT, AND ANALYSIS

(71) Applicant: Paulette Lawrence, Bloomfield, CT (US)

(72) Inventor: Paulette Lawrence, Bloomfield, CT (US)

(21) Appl. No.: 17/718,426

(22) Filed: Apr. 12, 2022

Related U.S. Application Data
(60) Provisional application No. 63/174,263, filed on Apr. 13, 2021.

(51) Int. Cl. G16H 40/20; G16H 20/10; G16H 10/60; G06Q 10/08

(52) U.S. Cl. CPC G16H 40/20; G16H 20/10; G16H 10/60; G06Q 10/087

(57) ABSTRACT

A centralized system comprising a control server with a vaccination verification module for creating patient vaccination records and providing proof of vaccination by anonymously referencing the vaccination records using unique patient identifiers, an inventory management module for monitoring vaccine distribution and automatically updating vaccine supply levels following administering of vaccine doses, and a reporting module for identifying distribution discrepancies and generating agency reports for governmental agencies.

### CROSS-REFERENCE TO RELATED APPLICATIONS

This application is a nonprovisional utility application of provisional patent application, Ser. No. 63/174,263 filed in the United States Patent Office on Apr. 13, 2021, claims priority therefrom, and is expressly incorporated herein by reference in its entirety.

### TECHNICAL FIELD

The present disclosure relates generally to a computer system for storing and verifying patient information and managing medical supply inventories. More particularly, the present disclosure relates to a centralized system for managing and tracking vaccine distribution and inventory, creating and maintaining vaccination records, and providing electronic proof of vaccination.

### BACKGROUND

Extensive vaccination of populations and observance of preventive behaviors such as social distancing by unvaccinated individuals are key to controlling the spread of highly infectious diseases during an epidemic or pandemic. Vaccinated individuals are unlikely to infect others with the pathogens against which they are vaccinated, and may safely reduce their observance of preventative behaviors, particularly when interacting with other vaccinated individuals.

There are various forms of vaccine credentials which allow vaccinated individuals to provide proof of vaccination to interested third parties such as employers, schools and universities, travel agencies, and businesses within the entertainment industry. Vaccination record cards distributed by the Centers for Disease Control (CDC) are one such example. However, such vaccine credentials do not prevent the use of falsified credentials by unvaccinated individuals. Although various electronic systems for providing verifiable proof of vaccination are found in the prior art, these systems lack standardization, and are unable to function as a centralized vaccination database.

Furthermore, during periods of widespread infection, effective vaccination strategies depend on the availability of accurate information pertaining to distribution of vaccines and vaccine supply levels which can help identify bottlenecks, supply shortages, or oversupply, thus facilitating coordinated cooperation between healthcare providers and governmental agencies. However, the lack of a centralized database prevents efficient sharing of information between healthcare providers, governmental agencies of different jurisdictions, and vaccine manufacturers.

A need therefore exists for a centralized system which is capable of not only providing authentic and verifiable proof of vaccination for individuals to interested third parties, but which is further capable of monitoring the vaccination process from distribution to dispensing, and providing centralized access to medically significant data.

In the present disclosure, where a document, act or item of knowledge is referred to or discussed, this reference or discussion is not an admission that the document, act or item of knowledge or any combination thereof was at the priority date, publicly available, known to the public, part of common general knowledge or otherwise constitutes prior art under the applicable statutory provisions; or is known to be relevant to an attempt to solve any problem with which the present disclosure is concerned.

While certain aspects of conventional technologies have been discussed to facilitate the present disclosure, no technical aspects are disclaimed and it is contemplated that the claims may encompass one or more of the conventional technical aspects discussed herein.

### BRIEF SUMMARY

An aspect of an example embodiment in the present disclosure is to provide an integrated system for recording the administering of vaccines to patients and providing proof of vaccination to third parties, while maintaining patient privacy. Accordingly, the present disclosure provides a centralized system comprising a plurality of user devices, and a control server having a vaccination verification module and a platform database. The centralized system has a plurality of users including medical users acting on behalf of the vaccination sites, patient users, and verification partner users, who access the centralized system using the user devices. The vaccination verification module allows the medical users to create vaccination records linked to patient profiles which confirm that a vaccine dose has been administered to one of the patients, and also generates a unique patient identifier for each patient which allows the vaccination record of the patient to be referenced anonymously. The patient identifier maintains patient privacy and reduces risk of identity theft by preventing exposure of sensitive information such as dates of birth, addresses, taxpayer registration numbers, and social security numbers. The vaccination verification module further allows the verification partner users to obtain proof of vaccination for one of the patients by submitting the patient identifier of the patient to the vaccination verification module, whereupon the vaccination verification module references the vaccination record using the patient identifier, and generates a vaccination verification report.

It is another aspect of an example embodiment in the present disclosure to provide an integrated system which allows manufacturers to track distribution of the vaccine doses to the vaccination sites, and vaccination sites to manage supplies of vaccine doses over the course of vaccination operations. Accordingly, the control server further has an inventory management module which maintains distribution records for manufacturers and inventory records associated with each of the vaccination sites, and further automatically updates the inventory records when vaccine doses are administered.

It is yet another aspect of an example embodiment in the present disclosure to provide an integrated system which allows governmental agencies to exercise oversight over the vaccination process. Accordingly, the control server further has a reporting module, allowing agency users acting on behalf of an agency to view the inventory records and vaccination records of all the vaccination sites within the agency's jurisdiction, and generate an agency report allowing medically significant trends to be identified. The agency reports may also incorporate census-level data for further analysis of vaccination-related trends.

The present disclosure addresses at least one of the foregoing disadvantages. However, it is contemplated that the present disclosure may prove useful in addressing other problems and deficiencies in a number of technical areas. Therefore, the claims should not necessarily be construed as limited to addressing any of the particular problems or deficiencies discussed hereinabove. To the accomplishment of the above, this disclosure may be embodied in the form illustrated in the accompanying drawings. Attention is called to the fact, however, that the drawings are illustrative only. Variations are contemplated as being part of the disclosure.

### BRIEF DESCRIPTION OF THE DRAWINGS

In the drawings, like elements are depicted by like reference numerals. The drawings are briefly described as follows.

FIG. 1 is a block diagram depicting a centralized system with a control server and a plurality of user devices, the control server having an access control module, an inventory management module, a reporting module, and a platform database, in accordance with an embodiment in the present disclosure.

FIG. 2 is a block diagram depicting an example architecture of the control server, in accordance with an embodiment in the present disclosure.

FIG. 3 is a block diagram depicting the access control module controlling access to the platform database according to a plurality of user access control rules, in accordance with an embodiment in the present disclosure.

FIG. 4A is a block diagram depicting an example distribution record and an example manufacturer profile, in accordance with an embodiment in the present disclosure.

FIG. 4B is a block diagram depicting an interaction between a manufacturer user and the centralized system, in accordance with an embodiment in the present disclosure.

FIG. 5A is a block diagram depicting an inventory tracking function for managing vaccine inventories at one of the vaccination sites, in accordance with an embodiment in the present disclosure.

FIG. 5B is a block diagram depicting an example site profile for one of the vaccination sites, in accordance with an embodiment in the present disclosure.

FIG. 6A is a block diagram showing an example process by which patient profiles and vaccination records are created and inventory records are updated, in accordance with an embodiment in the present disclosure.

FIG. 6B is a block diagram depicting an example vaccination record, in accordance with an embodiment in the present disclosure.

FIG. 6C is a block diagram depicting an example user device displaying a verification code which can be scanned to provide proof of vaccination, in accordance with an embodiment in the present disclosure.

FIG. 6D is a block diagram depicting an example of a physical vaccination certificate upon which the verification code is printed, in accordance with an embodiment in the present disclosure.

FIG. 7 is block diagram depicting a process by which a verification partner user obtains a vaccination verification report providing proof of vaccination for one of the patients, in accordance with an embodiment in the present disclosure.

FIG. 8 is a block diagram depicting an example process allowing agency users to access data records for all vaccination sites within a jurisdiction and obtain agency reports based on the jurisdictional level data, in accordance with an embodiment in the present disclosure.

FIG. 9 is a flowchart depicting an example vaccine operational process, in accordance with an embodiment in the present disclosure.

The present disclosure now will be described more fully hereinafter with reference to the accompanying drawings, which show various example embodiments. However, the present disclosure may be embodied in many different forms and should not be construed as limited to the example embodiments set forth herein. Rather, these example embodiments are provided so that the present disclosure is thorough, complete and fully conveys the scope of the present disclosure to those skilled in the art.

### DETAILED DESCRIPTION OF THE PREFERRED EMBODIMENTS

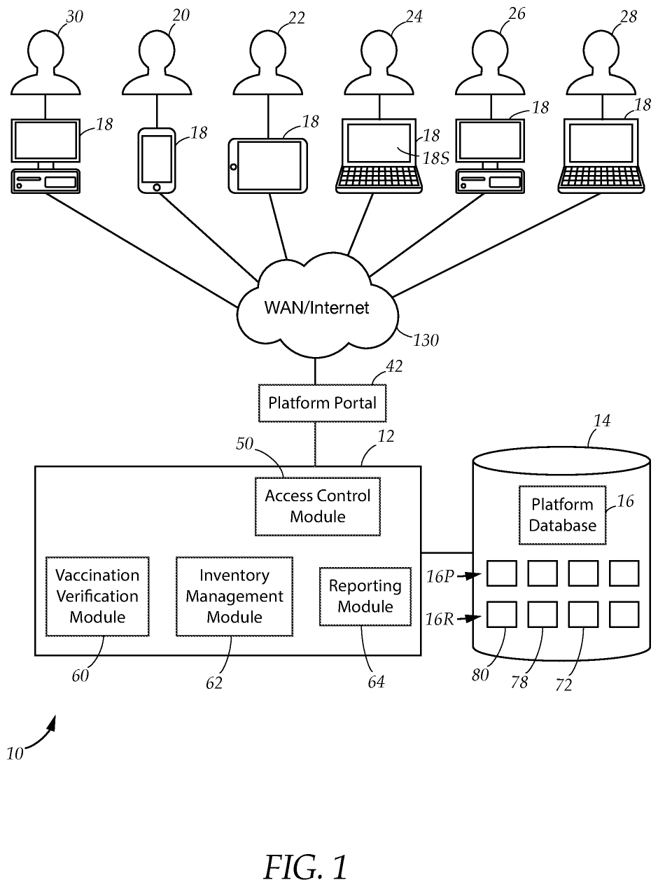

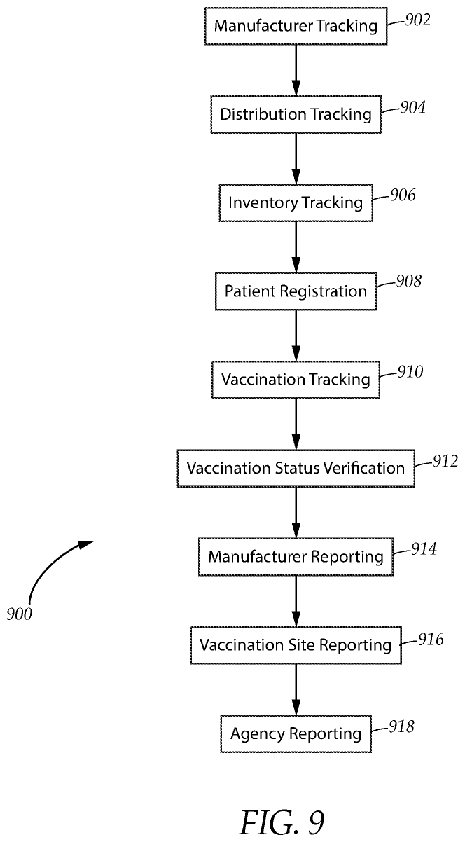

FIGS. 1 and 9 illustrate a centralized system 10 for implementing a vaccine operational process 900 for distributing and administering vaccines, and providing verifiable proof of vaccination status for vaccine recipients. The centralized system 10 comprises a control server 12, a platform database 16, and a platform portal 42. The control server 12 has a plurality of modules which carry out functions of the centralized system 10, comprising a vaccination verification module 60, an inventory management module 62, a reporting module 64, and an access control module 50. The modules are each adapted to execute one or more steps of the vaccine operational process 900.

Participants in the vaccine operational process 900 include manufacturers of the vaccines, vaccination sites at which the vaccines are administered, patients to whom the vaccines are administered, verification partners requesting proof of vaccination from patients, and agencies such as governmental organizations exercising oversight of the vaccine operational process 900. Participants in the vaccine operational process are represented as users of the centralized system 10, defined according to user types including manufacturer users 30 acting on behalf of the manufacturers, patient users 20, medical users 22 who operate the vaccination sites, verification partner users 28, and agency users 24. Each of the users accesses the centralized system 10 using one of a plurality of user devices 18. The control server 12 is operably connected to the user devices 18 via a data communication network 130, such as the Internet.

The platform database 16 contains a plurality of data records 16R and a plurality of user profiles 16P. The data records 16R embody information related to particular stages of the vaccine operational process 900, while the user profiles 16P are employed by the access control module 50 to store user data and determine the user platform functions available to the user, as well as the scope of the user's data access permissions.

Referring to FIG. 1 and FIG. 9, the vaccine operational process 900 comprises a series of monitored steps overseen by the centralized system 10, through which actions of the participants are facilitated or tracked. In one embodiment, the process 900 begins with a manufacturing tracking step 902 where one of the manufactures produces a portion of the vaccines, and the vaccines are organized into lots for subsequent tracking. The process 900 proceeds to a distribution tracking step 904, by which shipments of vaccines are distributed to vaccination sites. Next, an inventory tracking step 906 occurs, by which vaccine stocks received by the vaccination sites are organized and tracked. In a preferred embodiment, the inventory management module 62 is adapted to execute the manufacturing tracking 902, distribution tracking 904, and inventory tracking 906 stages of the vaccine operational process 900.

Continuing with the vaccine operational process 900, the vaccination verification module 60 is adapted to execute a patient registration step 908 during which patients are registered with the centralized system 10, and a vaccination tracking step 910 by which the patient's vaccination status is updated and stored. The vaccination verification module 60 further allows proof of the vaccination status to be retrieved at a vaccination status verification step 912.

The reporting module 64 provides the centralized system 10 with reporting capabilities which track distribution or inventory discrepancies across the stages of the vaccine operational process 900. The reporting module 64 is further adapted to identify medically significant trends within the information contained within the platform database 16. The reporting module 64 is therefore adapted to execute a manufacturer reporting step 914, a vaccination site reporting step 916, and an agency reporting step 918 in order to generate reports optimized for the needs of manufacturer users 30, medical users 22, and agency users 24 respectively.

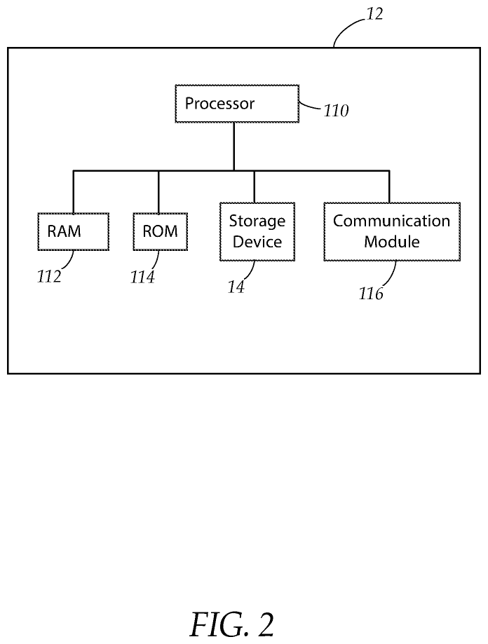

Turning to FIG. 2 while continuing to refer to FIG. 1, the inventory management module 62, the access control module 50, the vaccination verification module 60, and the reporting module 64 may be implemented using software components, packages, or assemblies which provide the functionality of the centralized system 10. The control server 12 is a computing device adapted to execute the computer program code of the various modules, as well as communicate with the user devices 18 via the data communication network 130. In one embodiment, the user devices 18 and the control server 12 operate in a client-server relationship, whereby requests to invoke functions of the centralized system are transmitted to the control server 12 by the user devices 18. The control server 12 is adapted to execute the requested functions, and transmit appropriate data responses to the requesting user devices 18. In one embodiment, the control server 12 has a processor 110, a RAM 112, a ROM 114, a computer storage device 14, and a communication module 116 adapted to communicate electronically via the data communication network 130 with the user devices 18 and other computing devices using a communications protocol. The files which form the platform database 16 can be stored using the computer storage device 14, or on a separate file server or database server which is accessible to the control server 12.

The user device 18 is a computer, tablet, mobile phone, or other computing device which is configured to communicate with the control server 12 via the data communication network 130. Each user device 18 has a device screen 18S for displaying information and graphical elements, and an input device for accepting user input.

In a preferred embodiment, the platform portal 42 provides each of the users with a set of user platform functions presented via the device screens 18S of the user devices 18. The platform portal 42 may be implemented through a web page, web application, or locally executed application running on the user device 18. A web-accessible variant of the platform portal 42 may be hosted via a separate web server, or the control server 12. Any user platform functions selected or initiated by the users are relayed to the control server 12.

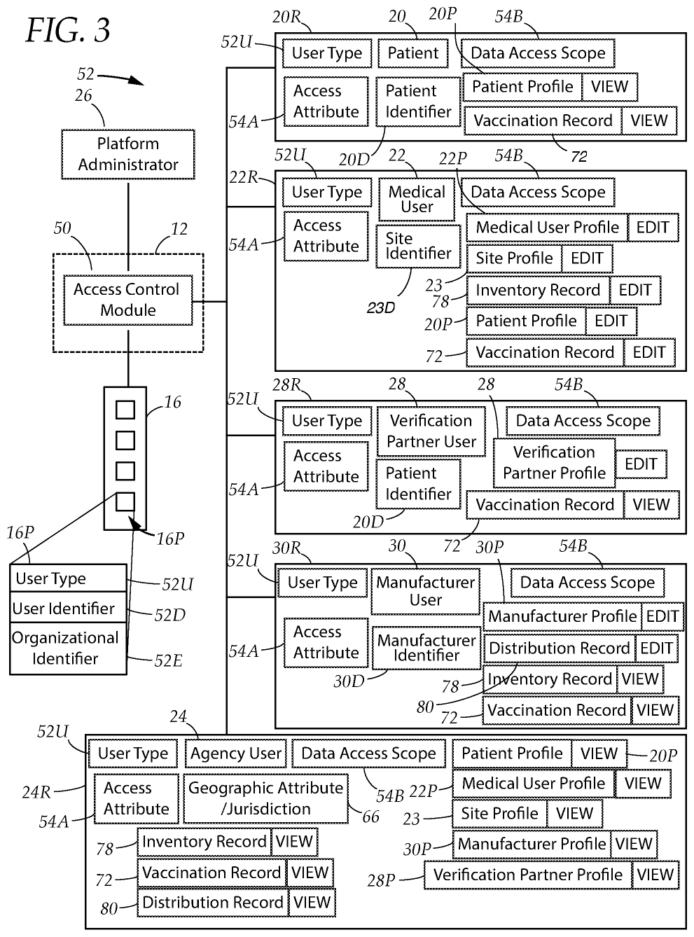

Turning to FIG. 3 while also referring to FIG. 1, the access control module 50 is adapted to selectively restrict or allow access of each user to the functions and data of the centralized system 10. Each user must have a valid user profile 16P in order to access the centralized system 10. Requests by users to register and create new user profiles 16P may be subject to approval by the platform administrator 26. The platform administrator 26 may also directly initiate creation of new user profiles 16P, with each user completing the registration by logging in using appropriate login credentials. In certain embodiments, the centralized system 10 may allow agency users 24 to act as platform administrators 26, thus allowing agency users 24 to approve registration of vaccination sites, medical users 22, and manufacturer users 30.

The access control module 50 may be configured to add the newly created user profiles 16P to the platform database 16. In one embodiment, each user profile 16P contains a plurality of user attributes which define relevant user characteristics, and which are utilized by the access control module 50 to determine the user's access to functions and data. The user attributes may comprise a user type 52U describing the user's role, as well as a user identifier 52D which uniquely identifies the user. Certain user profiles 16P may also contain an organizational identifier 52E, which identifies a participant who is not a person, such as a manufacturer, vaccination site, or agency with which the user is affiliated.

The access control module 50 has a plurality of access control rules 52, with each access control rule 52 specifying a user type 52U, as well as a data access scope 54B. The access control rule 52 defines the user platform functions which are available to each user of the user type 52U, while the data access scope 54B identifies the data records 16R and user profiles 16P which are accessible to said users. The data access scope 54B further specifies whether the user is allowed to view or edit the identified data records 16R or user profiles 16P.

In certain embodiments, the access control rules 52 may specify one or more access attributes 54A which further limit the data access scope 54B. For example, data records 16R may be linked to specific user identifiers 52D or organizational identifiers 52E. The access attribute 54A may therefore be used to restrict the data access scope 54B to only data records 16R which share the same organizational identifier 52E or user identifier 52D as the accessing user.

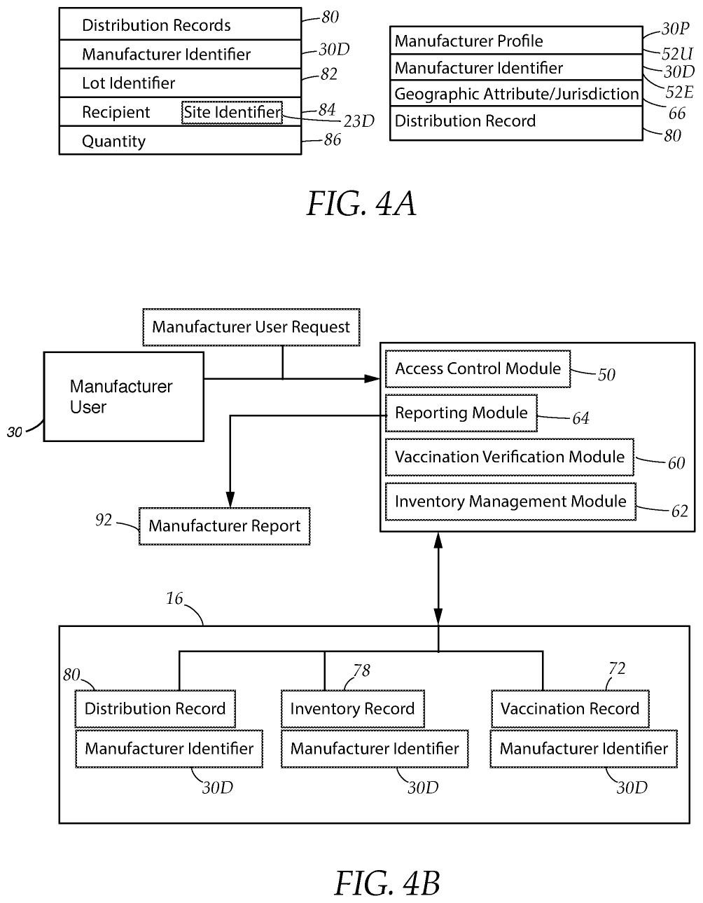

Turning to FIGS. 4A-B while also referring to FIG. 1, FIG. 3, and FIG. 9, vaccines produced by a manufacturer are registered at the manufacturing tracking step 902 for subsequent tracking throughout the vaccine operational process 900. In one embodiment, vaccines and vaccine boosters are organized into lots comprising a quantity of vaccine doses. Each lot is represented by a distribution record 80 which is created via the inventory management module 62 and is stored within the platform database 16. The distribution record 80 contains a lot identifier 82 which uniquely identifies the lot, and may further contain the manufacturer identifier 30D, thus linking the distribution record 80 to the manufacturer.

The vaccine doses within each lot may be distributed to one or more recipients, typically corresponding to vaccination sites. The distribution record 80 may therefore contain a recipient field 84 for each recipient to which vaccine doses from the lot are distributed, along with a quantity field 86 indicating the total quantity of vaccine doses from the lot which have been sent to the recipient. When the recipient is a vaccination site, the recipient field 84 may be populated by the site identifier 23D which uniquely identifies the vaccination site. Recording of the recipient field 84 and quantity field 86 occurs at the distribution tracking step 904.

In one embodiment, each distribution record 80 is created and updated through user platform functions accessible to manufacturer users 30. Each manufacturer user 30 is linked to a manufacturer profile 30P.

The example manufacturer profile 30P contains a user type 52U condition indicating that the rule applies to manufacturer users 30, and may further contain the manufacturer identifier 30D as an organizational identifier 52E attribute. An example manufacturer access control rule 30R contained within the access control rules 52 is shown, which allows a manufacturer user 30 to view, create, and edit distribution records 80. The manufacturer access control rule 30R may limit the manufacturer user 30 to accessing data records 16R that share the same manufacturer ID 30D as the manufacturer user 30, thus preventing the manufacturer user 30 from accessing distribution records 80 linked to a different manufacturer.

Upon logging in to the centralized system 10, the manufacturer user 30 is presented with the appropriate user platform functions via the platform portal 42. As each new lot is produced, the manufacturer user 30 may submit a manufacturer user request to register the lot with the centralized system 10. As vaccine doses are distributed to recipients, the manufacturer user 30 may submit another manufacturer user request to update the distribution record 80 and record the recipient and the quantity of vaccine doses which are sent to the recipient. The manufacturer user requests are approved or denied by the access control module 50 in accordance with the manufacturer access control rule 30R, and the approved manufacturer user requests are carried out by the inventory management module 62.

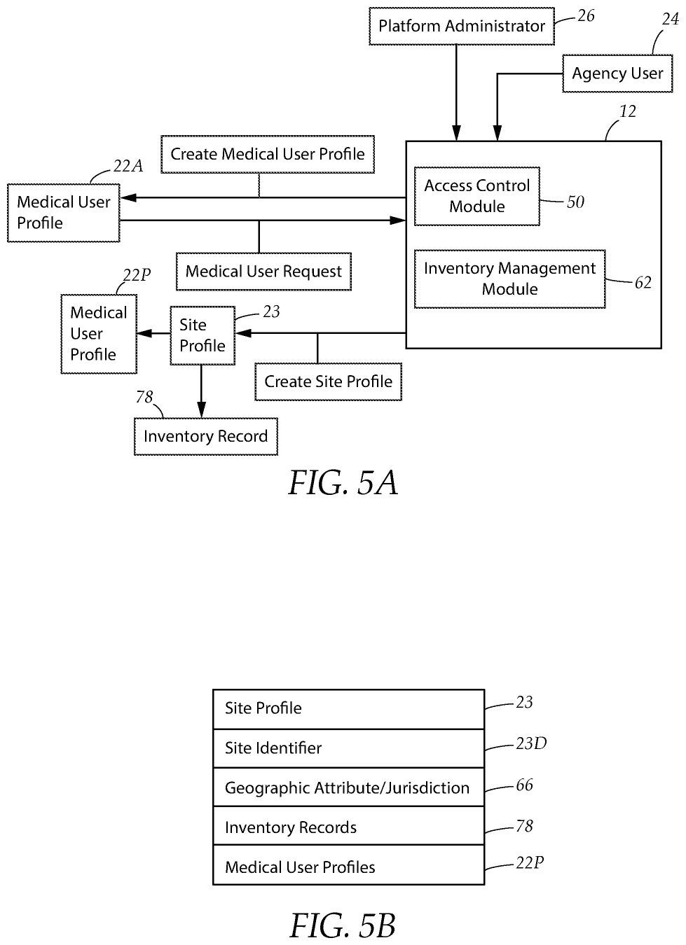

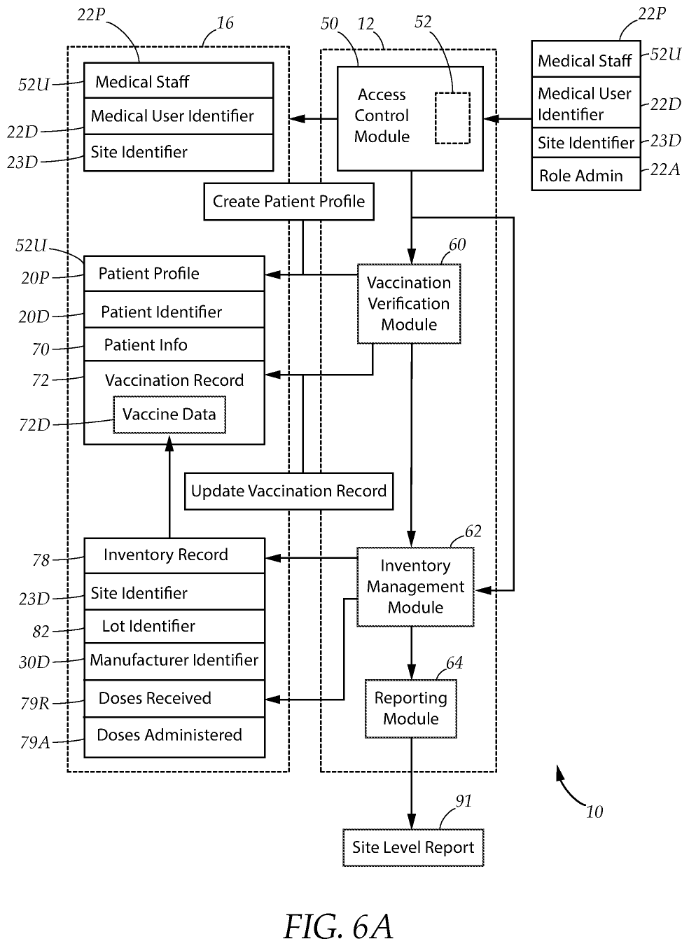

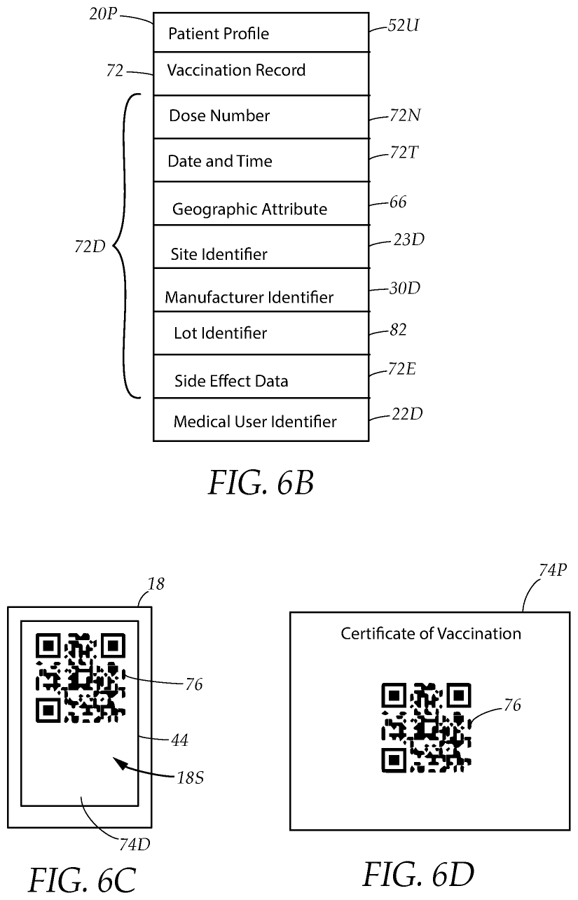

Turning to FIGS. 5A-B and FIG. 6A while also referring to FIG. 1, FIG. 3, and FIG. 9, during the inventory tracking step 906 of the vaccine operational process 900, the inventory management module monitors inventories of vaccine doses which have been distributed to vaccination sites. Vaccine doses which are allocated to, or which are present at a vaccination site, are registered with the centralized system 10 via an inventory record 78 stored within the platform database 16. The inventory record 78 is used to track a vaccine supply level, whereby the quantity of vaccine doses associated with each vaccination site increases following receipt of new vaccine doses, and decreases following administration of vaccine doses to patients. The inventory records 78 may also be used to link the vaccine doses to the lot and the manufacturer which produced the vaccine.

In a preferred embodiment, each vaccination site is associated with a site profile 23 stored within the platform database 16. The site profile 23 contains the site identifier 23D of the vaccination site, and may also be linked to the medical user profiles 22P of any medical users 22 who are affiliated with the vaccination site. In one embodiment, new site profiles 23 may be created using user platform functions available to medical users 22, platform administrators 26, and agency users 24.

Turning to FIG. 6A while also referring to FIG. 1, FIG. 3, FIG. 5A, and FIG. 9, an exemplary medical user access control rule 22R grants each medical user 22 access to the user platform functions and data access scope 54B necessary to operate the vaccination site. The medical user profile 22P of each medical user 22 may include a medical user identifier 22D which uniquely identifies the medical user 22, as well as an organizational identifier 52E which corresponds to the site identifier 23D of the vaccination site with which the medical user 22 is affiliated.

The medical user access control rule 22R specifies a user type 52U condition which indicates that the rule 22R applies to medical users 22. The data access scope 54B of the rule 22R may indicate that the medical user 22 is authorized to view, create, and edit site profiles 23, inventory records 78, patient profiles 20P, as well as medical user profiles 22P. In order to prevent medical users 22 from accessing data which is unrelated to the vaccination site to which each medical user 22 is affiliated with, the rule 22R may include an access attribute 54A condition which permits access only to those data records 16R and user profiles 16P which contain or which are linked to the same site identifier 23D contained within the medical user profile 22P of the accessing medical user 22.

Upon logging in to the centralized system 10, the medical user 22 is presented with the appropriate user platform controls by the platform portal 42, in accordance with the medical user access control rule 22R. The inventory records 78 of each vaccination site are created and edited through medical user requests submitted to the inventory management module 62 by the medical users 22 affiliated with the particular vaccination site. Upon receiving new vaccine doses from one of the manufacturers, the medical user 22 may submit a medical user request to the inventory management module 62 to create or update an inventory record 78 containing the site identifier 23D of the vaccination site. The new vaccine doses are embodied within the inventory record 78 by the lot identifier 82 and the manufacturer identifier 30D, and the quantity of the new vaccine doses received is reflected via doses received attribute 79R. A doses administrated attribute 79A may be used to record the quantity of doses which are administered to patients.

Referring to FIGS. 6A-B while also referring to FIG. 1, FIG. 3, and FIG. 9, the patient registration step 908 allows new patients to be registered with the centralized system 10 in preparation to receive the vaccine. In a preferred embodiment, upon a patient visiting one of the vaccination sites, one of the medical users 22 affiliated with the vaccination site submits a medical user request to the vaccination verification module 60 to create a new patient profile 20P. The patient profile 20P contains a patient identifier 20D attribute, which is populated only if the vaccine is administered to the patient. The patient profile 20P may also be used to store patient information 70, such as personally identifying information and medical information.

At the vaccination tracking step 910 of the vaccine operational process 900, one of the vaccine doses under the control of the vaccination site is administered to the patient. As specified by the medical user access control rule 22R, the medical user 22 is permitted to view, create, and edit vaccination records 72. One of the medical users 22 may submit a vaccination confirmation command to the vaccination verification module 60 via the user device 18, causing the vaccination verification module 60 to create a new vaccination record 72 which is linked to the patient profile 20P and is stored within the platform database 16. Each vaccination record 72 provides confirmation that the patient user 20 associated with the vaccination record 72 has received at least one dose of the vaccine.

Once the medical user request to create the vaccination record 72 is submitted, the vaccination verification module 60 will generate a unique alphanumeric sequence which will be used to populate the patient identifier 20D attribute within the patient profile 20P. Each patient identifier 20D is unique, and allows patient profiles 20P and vaccination records 72 to be referenced anonymously without using personally identifiable information.

The vaccination record 72 may contain dosage data 72D which identifies the vaccination site, the manufacturer of the vaccine dose, the lot from which the administered vaccine dose was drawn, as well as the date and time at which the vaccine dose is administered to the patient. For vaccines which require administration of more than one vaccine dose, the dosage data 72D may also include a dose number 72N attribute which tracks the number of vaccine doses administered to the patient, including vaccine booster shots where applicable. After the vaccination confirmation command is submitted, the vaccination verification module 60 creates the vaccination record 72, and populates the vaccination record 72 with the appropriate site identifier 23D, manufacturer identifier 30D, lot identifier 82, and time data 72T. The site identifier 23D, manufacturer identifier 30D, or the lot identifier 82 may be submitted with the vaccination confirmation command. In certain embodiments, the vaccination record 72 may also contain the medical user identifier 22D of the medical user who administered the vaccine dose to the patient. In a preferred embodiment, the vaccination record 78 does not contain sensitive personal information of the patient user 20, and the patient identifier 20D serves to link the patient profile 20P to the vaccination record 72.

In one embodiment, the time data 72T field is a static field which is populated in real-time when the vaccination record 72 is initially created by the vaccination verification module, and cannot be altered at the request of any user. The static time data field 72T therefore functions as a countermeasure against attempts to fraudulently or erroneously alter vaccination records 72. In certain embodiments, the vaccination verification module 60 may be configured to detect subsequent attempts to alter, edit, or delete a vaccination record 72, and generate a security alert or security report which is presented to one of the medical users 22 or a platform administrator 26.

The inventory management module 62 automatically updates the relevant inventory record 78 after the administering of each vaccine dose to reflect the reduction in the quantity of the vaccine doses available to the vaccination site. In a preferred embodiment, the creation of the vaccination record 72 causes the inventory management module 62 to update the inventory record 78 of the vaccination site associated with the vaccination record 72. In one embodiment, the site identifier 23D and the lot identifier 82 of the administered vaccine dose may be used to reference the appropriate inventory record 78, and the inventory management module 62 adjusts the inventory record 78 to reflect the reduction in the vaccine supply level. In one embodiment, the doses administered attribute 79A of the inventory record 78 is increased. To determine the quantity of vaccine doses remaining at a vaccination site, the value of the doses administered 79A field may simply be subtracted from the value of the doses received 79R field.

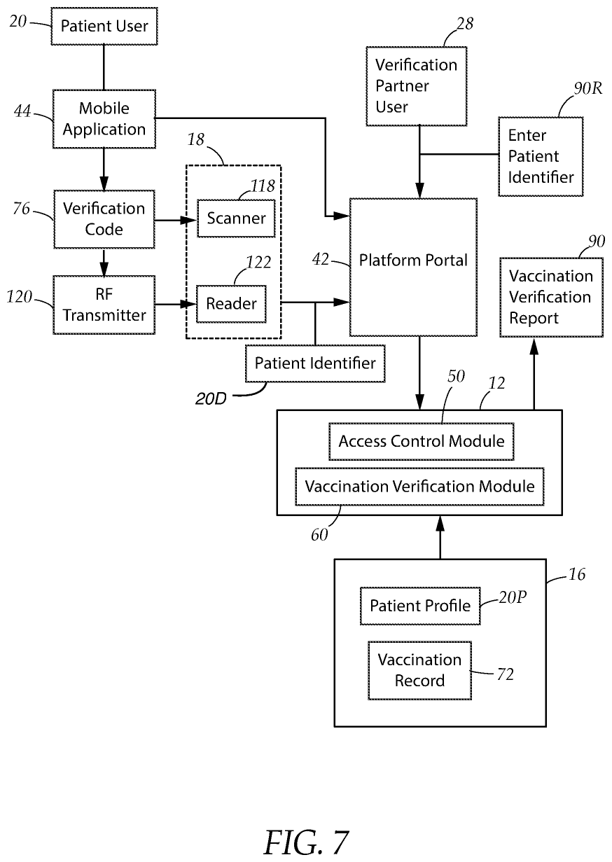

Referring to FIG. 7 while also referring to FIG. 1, FIG. 3, FIG. 6A-D, and FIG. 9, the centralized system 10 is adapted to provide proof of vaccination at the vaccination status verification step 912 of the vaccine operational process 900. Verification partners are typically businesses, individuals, or organizations, or entities with an interest in confirming the vaccination status of a patient user 20. The centralized system 10 allows authenticated verification partner users 28 to view a vaccination verification report 90 which confirms the vaccination status of a patient user 20, without requiring the patient user 20 to reveal any information beyond the anonymized patient identifier 20D.

In a preferred embodiment, the patient identifier 20D is embodied within a machine-readable verification code 76. The verification code 76 may be in the form of an optical code such as a QR code, bar-code, or other machine-readable optical code format. The verification partner user 28 employs a user device 18 equipped with an optical scanner 118 to scan the verification code 76, read the patient identifier 20D, and submit a vaccination verification report request 90R to the centralized system 10 containing the scanned patient identifier 20D.

In one embodiment, the patient user 20 may display the verification code 76 through a mobile application 44 implemented on a user device 18. A patient user 20 may access the centralized system 10 through the mobile application 44 and be authenticated using the patient identifier 20D. Once authenticated, the patient user 20 may use the mobile application 44 to display the verification code 76. For example, the verification code 76 may be displayed in QR code format on the device screen 18S of the user device 18 of the patient user 20. The user device 18 may be a mobile phone, tablet, wearable computing device such as a smartwatch, or any portable computing device capable of communicating with the control server 12 via the data communication network 130.

Note that in certain embodiments, the verification code 76 may instead be transmitted using an RF transmitter 120. The RF transmitted 120 may be an NFC chip integrated within a user device 18, an RFID tag, or other transmitting device capable of transmitting data via a wireless data communication protocol as will be apparent to a person of ordinary skill in the art in the field of the invention. The user device 18 of the verification partner 28 will correspondingly be equipped with an RF reader 122 configured to receive and interpret signals to read the verification code 76.

Alternatively, the patient user 20 may be provided with a physical vaccination certificate 74P, which incorporates the verification code 76 in printed form. The physical vaccination certificate 74P may be a card, a paper certificate, or other tangible piece of material upon which the verification code 76 may be placed in a manner which allows the verification code 76 to be scanned by an optical scanner 118. A physical vaccination certificate 74P may be issued to the patient user 20 after the patient user 20 receives the vaccination dose and the vaccination record 72 has been created. For example, physical vaccination certificates 74P may be issued at vaccination sites to patients following the administering of vaccine doses.

In certain embodiments, in lieu of scanning the machine readable verification code 76, the platform portal 42 may allow the verification partner user 28 to manually enter the patient identifier 20D in order to complete the vaccination verification report verification request.

After receiving the vaccination verification report request 90R, the vaccination verification module 60 retrieves the vaccination record 72 associated with the submitted patient identifier 20D, and generates a vaccination verification report 90. In the interest of privacy and data security, the access control module 50 restricts a verification partner user 28 to accessing only the minimum data necessary to verify the vaccination status of the patient user 20 associated with the patient identifier 20D. Therefore, the access control module 50 may employ a verification partner access control rule 28R containing a data access scope descriptor 54B which allows a verification partner user 28 to access vaccination records 72, along with a data access attribute 54A corresponding to the patient identifier 20D which is encoded within the verification code 76.

The vaccination verification report 90 is displayed using the user device 18 of the verification partner user 28, and includes relevant data included in the vaccination record 72 sufficient to confirm the vaccination status of the patient user 20. For example, the vaccination verification report 90 may display dosage data 72D, such as the quantity of the vaccine doses received by the patient user 20, along with the date and time at which each of the vaccine doses was administered.

Referring to FIGS. 4A-B while also referring to FIG. 1, FIG. 3, FIGS. 6A-B, and FIG. 9, the manufacturer reporting step 914 allows manufacturer users 30 to request a manufacturer distribution report 92 from the reporting module 64 which compares the total quantity of vaccine doses distributed to vaccination sites, to the total quantity of vaccine doses administered by the vaccination sites as well as the total quantity of vaccine doses held within the inventories of the vaccination sites. Manufacturer distribution reports 92 may utilize data from any data records 16R which the manufacturer user 30 may access as specified by the manufacturer user access control rule 30R. For example, the reporting module 64 may retrieve for analysis all distribution records 80 and inventory records 78 which are linked to a manufacturer by manufacturer identifier 30D and/or by lot identifier 82. The manufacturer distribution report 92 may identify site-level distribution discrepancies by comparing the quantity 86 of vaccination doses distributed to one of the vaccination sites as indicated in the distribution record 80, against the doses received attribute 79R contained in the inventory record 78 of the vaccination site, whereby a mismatch between the quantity 86 of vaccination doses distributed and the doses received 79R, or the quantity of doses received 79R combined with the quantity of doses administered, indicates a distribution discrepancy has occurred and vaccination doses are unaccounted for. The manufacturer distribution report 92 may also contain anonymous data retrieved from vaccination records 72 linked to vaccination doses manufactured by the manufacturer, as indicated by the manufacturer identifier 30D or lot identifier 82 which may be present within the vaccination records 72.

Turning to FIGS. 6A-B while also referring to FIG. 1, FIG. 3, and FIG. 9, the vaccination site reporting step 916 allows medical users 22 to request site level reports 91 from the reporting module 64 to analyze any aspect of the vaccine operational process 900 related to the vaccination site and to its patients. Site level reports 91 may include data from any data records 16R and user profiles 16P accessible to medical users 22 as specified by the medical user access control rule 22R, such as data from any medical user profiles 22P, site profiles 23, inventory records 78, patient profiles 20P, and vaccination records 72 which are associated with the site identifier 23D of the vaccination site or which is included in the organizational identifier 52E linked to the medical user 22 submitting the request for the site level report 91.

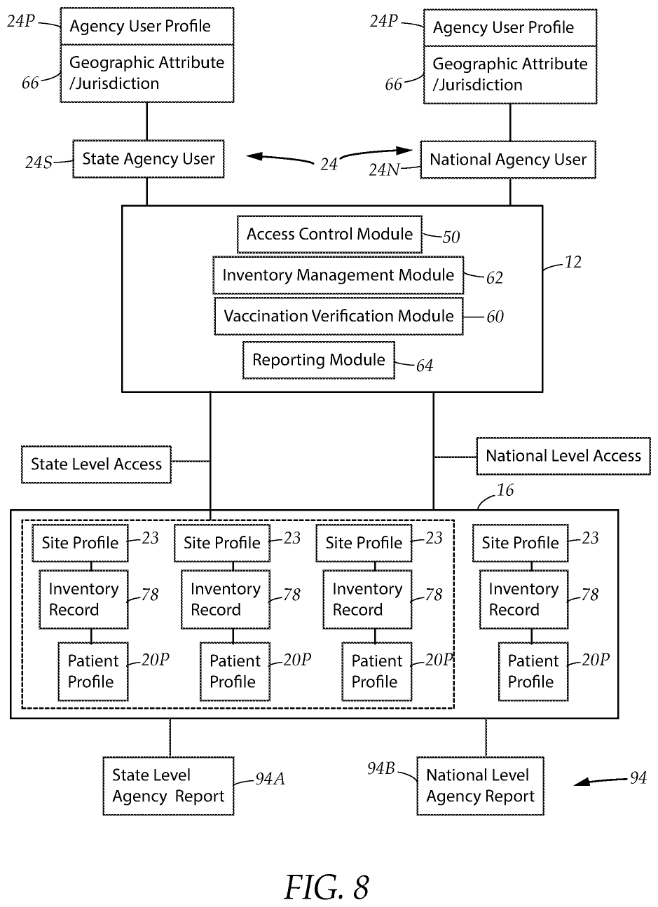

Referring to FIG. 1, FIG. 4B, FIGS. 6A-B, and FIG. 8, in certain embodiments, the platform portal 42 may also allow medical users 22 and patient users 20 to submit side effect information experienced by the patient user 22 following administration of the vaccination dose. Such submitted information may be recorded as side effect data 72E stored within individual vaccination records 72, and may be accessed by any medical users 22 as part of a site level report 91 or by manufacturer users 30 as part of a manufacturer distribution report 92, thus facilitating anonymous data for research into vaccination side effects. Furthermore, the reporting module 64 is adapted to trace the manufacturer or lot number of any vaccine dose associated with the side effect data 72E by referencing the manufacturer identifier 30D or the lot identifier 82 contained within the vaccination record 72, and include such tracing data within site-level reports 91, agency reports 94, distribution reports 92, or a separate side effect report.

Turning to FIG. 8 while also referring to FIG. 1, FIG. 3, and FIG. 9, the agency reporting step 918 allows agency users 24 to view data which allows government agencies to exercise oversight over the vaccine operational process 900. The centralized system 10 allows agency users 24 to view a broad range of data, including the data records 16R or user profiles 16P which are accessible to medical users 22, and in certain embodiments, to manufacturer users 30. For example, an agency user access control rule 24R may have a data access scope 54B which allows agency users 24 to view distribution records 80, inventory records 78, patient profiles 20P, and vaccination records 72. In a preferred embodiment, an agency is associated with a geographic attribute 66 which corresponds to an area of jurisdiction over which the agency exercises governmental oversight. The reporting module 64 is therefore adapted to generate an agency report 94 which identifies high-level trends within the jurisdiction over which the agency exercises oversight. An agency report 94 may also include running totals quantifying the number of vaccine doses administered, or the total number of vaccinated patients, within a jurisdiction.

For example, an agency with a national level jurisdiction, such as the CDC, has a geographic attribute 66 which corresponds to an entire country. An agency such as a Department of Health for a state or province, has a geographic attribute 66 corresponding to the state. Agency users 24 have agency profiles 24P which include the geographic attribute 66 of the agency on behalf of which they are acting. The agency user access control rule 24R therefore includes an access attribute 54A which grants access to data records 16R and user profiles 16P which are linked to, or which originate in, a jurisdiction which is encompassed by the geographic attribute 66 of the geographic attribute 66 of the requesting agency. A state-level agency report 94A may encompass any data originating within a particular state. A national-level agency report 94B, will have access to all data which is available to state level agencies.

Referring to FIGS. 4A-B, FIG. 5B and FIGS. 6A-B while continuing to refer to FIG. 1, FIG. 3, and FIG. 8, vaccination site profiles 23 may include a geographic attribute 66 which identifies the jurisdiction in which the vaccination site is physically located. Patient users 20 and their patient profiles 20P and vaccination records 72 may be linked to a particular jurisdiction via a geographic attribute 66 stored within the vaccination record 72 identifying the jurisdiction within which the vaccine dose was administered, or by referencing the geographic attribute 66 of the appropriate vaccination site.

By allowing an agency report 92 to access to all inventory records 78 and vaccination records 72 linked to a jurisdiction, the reporting module 64 is adapted to perform a variety of analyses, including identifying vaccination rates of populations within geographical regions, identifying surpluses or shortages of supplies of vaccine doses, spotting side effects, or identifying other trends of medical significance, as will be apparent to a person of ordinary skill in the art in the field of the invention. The reporting module 64 may utilize census level data to assist in the identification of medically significant trends for inclusion in the agency reports 92.

Similarly, agency reports 94 may include data from manufacturer distribution records 80, thus allowing agencies to monitor for inventory or distribution discrepancies, identify distribution or production bottlenecks or inefficiencies, as well as other trends which may be identified through analysis of the relevant data.

As will be appreciated by one skilled in the art, aspect of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate or transport a program for use by or in connection with an instruction execution system, apparatus or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. Other types of languages include XML, XBRL and HTML5. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described below with reference to flowchart illustrations and/or block diagrams of methods, apparatus (systems) and computer program products according to embodiments of the disclosure. Each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality and operation of possible implementations of systems, methods and computer program products according to various embodiments of the present disclosure. In this regard, each block in the flowchart or block diagrams may represent a module, segment or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiment was chosen and described in order to best explain the principles of the disclosure and the practical application, and to enable others of ordinary skill in the art to understand the disclosure for various embodiments with various modifications as are suited to the particular use contemplated.

The flow diagrams depicted herein are just one example. There may be many variations to this diagram or the steps (or operations) described therein without departing from the spirit of the disclosure. For instance, the steps may be performed in a differing order and/or steps may be added, deleted and/or modified. All of these variations are considered a part of the claimed disclosure.

In conclusion, herein is presented a centralized system for implementing a vaccine operational process. The disclosure is illustrated by example in the drawing figures, and throughout the written description. It should be understood that numerous variations are possible, while adhering to the inventive concept. Such variations are contemplated as being a part of the present disclosure.

What is claimed is:

1. A centralized system for controlling supplies of vaccine doses available to each of a plurality of vaccination sites, tracking administration of one of the vaccine doses to a patient of one of the vaccination sites, and providing proof of vaccination, the centralized system comprising:
a plurality of user devices, each user device is operated by one of a plurality of users each having a user type, the user types comprising a medical user, a verification partner user, and a patient user, each patient user corresponding to one of the patients;
a control server having an inventory management module, a vaccination verification module, and a platform database, the platform database containing a plurality of data records and user profiles;
the vaccination verification module is adapted to create and update a patient profile associated with one of the patient users, the patient profile corresponds to one of the user profiles, the vaccination verification module is further adapted to create a unique patient identifier and a vaccination record linked to the patient profile upon receiving a vaccination confirmation command from the user device operated by the medical user, the vaccination record confirms the administration of the vaccine dose to the patient and corresponds to one of the data records;
the patient identifier is encoded with a machine-readable verification code;
the inventory management module is adapted to perform an inventory tracking function by maintaining an inventory record associated with each of the vaccination sites, each inventory record corresponds to one of the data records and quantifies the vaccine doses available to the vaccination site, the tracking function allows the inventory management module to modify the inventory record to reduce the quantity of vaccine doses available to the vaccination site in response to the creation of the vaccination record;
the user device of the verification partner has a scanner adapted to read the verification code and interpret the patient identifier, allowing said user device to submit a verification request to the vaccination verification module which contains the patient identifier; and
the vaccination verification module is further adapted to authenticate the verification request by referencing the patient profile using the patient identifier, and present a vaccination verification report via the user device of the verification partner user.

2. The centralized system as described in claim 1, wherein:
the verification code is an optical code, and the scanner is an optical scanner adapted to scan and interpret the verification code; and
the vaccination verification module is further adapted to retrieve the verification code containing the patient identifier upon receiving a patient request submitted via the user device operated by the patient user, allowing the user device of the patient user to present the verification code for scanning by the user device of the verification partner user.

3. The centralized system as described in claim 2, wherein:
the vaccine doses are produced in lots by a manufacturer, and each lot is associated with a lot identifier;
each inventory record further contains the quantity of the vaccine doses received from the manufacturer;
the inventory management module is further adapted to perform a manufacturer distribution tracking function by maintaining a distribution record associated with the manufacturer, the distribution record corresponds to one of the data records and quantifies the vaccine doses dispatched to a recipient corresponding to one of the vaccination sites, the distribution record contains the lot identifier of the vaccine doses quantified by the distribution record.

4. The centralized system as described in claim 3, wherein:
the vaccine verification module is adapted to record the lot identifier of the vaccine dose administered to the patient within the vaccination record; and
the inventory management module is further adapted to reference the inventory record using the lot number of the vaccine dose administered to the patient.

5. The centralized system as described in claim 4, wherein:
the control module further has an access control module with a plurality of user access control rules, each user access control rule limits access by the users to the platform database according to the user type.

6. The centralized system as described in claim 5, wherein:
each medical user is linked to one of the vaccination sites, and each vaccination record is associated with the vaccination site at which the vaccine dose is administered; and
the user access control rules comprising a medical user access control rule, the medical user access control rule allowing each medical user to view and edit the inventory records, patient profiles, and vaccination records associated with the vaccination site to which the medical user is linked.

7. The centralized system as described in claim 6, wherein:
the control server further comprises a reporting module, the reporting module is adapted to generate a distribution discrepancy report comparing the distribution record with the inventory record of the recipient vaccination site to identify a discrepancy between the quantity of the vaccine doses distributed and the quantity of the vaccine doses received.

8. The centralized system as described in claim 7, wherein:
the user types further comprise an agency user, each agency user is associated with a jurisdiction, and each vaccination site is associated with a geographical attribute;
the user access control rules further comprise an agency user access control rule allowing each agency user to view the inventory records, patient profiles, and vaccination records of the vaccination sites within the jurisdiction of the agency user as indicated by the geographical attribute of each vaccination site; and
the reporting module is further adapted to generate an agency report analyzing the inventory records and vaccination records available to the agency user.

9. The centralized system as described in claim 8, wherein:
each vaccination record contains a timestamp generated in real-time as each vaccination record is created, and the vaccination verification module is adapted to prevent any of the users from modifying the timestamp.

10. The centralized system as described in claim 9, wherein:
the reporting module is adapted to allow the medical users to submit side effect data for recording within the vaccination records, and is further adapted to identify the lot of the vaccine dose associated with the side effect data by referencing the lot identifier contained within the vaccination record.

11. A method for controlling supplies of vaccine doses available to each of a plurality of vaccination sites, tracking administration of one of the vaccine doses to a patient of one of the vaccination sites, and providing proof of vaccination, comprising the steps of:
providing a control server having an access control module, an inventory management module, a vaccination verification module, and a platform database, the platform database containing a plurality of data records and user profiles, the control server is adapted to communicate with a plurality of user devices via a data communication network;
defining a plurality of user types describing a plurality of users, the user types comprising a medical user, a verification partner user, and a patient user, each patient user corresponding to one of the patients;
associating each medical user with a medical user profile stored within the platform database as one of the user profiles, and linking the medical user profile to one of the vaccination sites;
creating an inventory record within the platform database using the inventory management module for each of the vaccination sites by the inventory management module, storing a vaccine doses received field within the inventory record quantifying the vaccine doses received by the vaccination site, each inventory record tracking a vaccine supply level available to the vaccination site;
creating a patient profile for each of the patients using the vaccination verification module, and storing the patient profiles within the platform database as part of the user profiles;
submitting a vaccination confirmation command for one of the patients to the vaccination verification module by one of the medical users via one of the user devices;
creating a vaccination record by the vaccination verification module and storing the vaccination record within the platform database as one of the data records following the vaccination confirmation command, the vaccination record confirming the administration of the vaccine doses to the patient;
generating a unique patient identifier for the patient linking the vaccination record to the patient profile of the patient, encoding the patient identifier in a machine-readable verification code;
updating the inventory record associated with the vaccine dose administered to the patient using the inventory management module, and reducing the vaccine supply level of the inventory record;
scanning the verification code by the verification partner user using a scanner, and submitting a vaccination verification report request to the vaccination verification module by the verification partner user via one of the user devices, the vaccination verification report request containing the patient identifier; and
referencing the vaccination record associated with the patient identifier by the vaccination verification module, generating a vaccination verification report, and presenting the vaccination verification report to the user device operated by the verification partner user.

12. The method as recited in claim 11, wherein:
the step of scanning the verification code is preceded by the step of submitting a patient request to the vaccination verification module by one of the patient users via one of the user devices, displaying the verification code in a machine-readable optical format via a device screen of said user device.

13. The method as recited in claim 12, wherein:
the vaccine doses are produced in lots by a manufacturer;
the step of creating an inventory record is preceded by the step of:
creating a distribution record associated with the manufacturer using the inventory management module and storing the distribution record within the platform database as one of the data records, storing a vaccine doses dispatched field within the distribution record quantifying the vaccine doses distributed to a recipient corresponding to one of the vaccination sites, associating each of the lots with a lot identifier, and storing the lot identifier associated with the vaccine doses quantified by the distribution record.

14. The method as recited in claim 13, wherein:
the step of creating an inventory record further comprises storing the lot identifier of each of the vaccine doses quantified by the inventory record;
the step of submitting a vaccination confirmation command further comprises submitting the lot identifier of the vaccine dose administered to the patient;
the step of creating a vaccination record further comprises storing the lot identifier within the vaccination record; and
the step of updating the inventory record further comprises updating the inventory record associated with the vaccine dose administered to the patient using the inventory management module by referencing the inventory record using the lot identifier contained within the vaccination verification command.

15. The method as recited in claim 14, wherein:
the control module further comprises an access control module; and
the step of defining a plurality of user types is followed by the step of defining a plurality of user access control rules limiting access by the users to the platform database according to user type.

16. The method as recited in claim 15, wherein:
the step of defining a plurality of user access control rules is followed by the step of defining a medical user access control rule allowing each medical user to view and edit the data records and user profiles associated with the vaccination site to which the medical user is linked; and
the step of creating a vaccination record further comprises associating the vaccination record with the vaccination site at which the vaccine dose is administered;

17. The method as recited in claim 16, wherein:
the control server further comprises a reporting module;
the step of updating the inventory record is followed by the step of generating a distribution discrepancy report by the reporting module comparing the distribution record with the inventory record of the recipient vaccination site, and identifying a discrepancy between the vaccine doses dispatched field and the vaccine doses received field.

18. The method as recited in claim 17, wherein:
the step of defining a plurality of user types further comprises defining an agency user as one of the user types;
the step of associating each medical user with a medical user profile is followed by the steps of:
associating each agency user with a jurisdiction, associating each of the vaccination sites with a geographical attribute encompassed within one of the jurisdictions; and defining an agency user access control rule allowing each agency user to view the data records and user profiles associated with each vaccination site encompassed within the jurisdiction of the agency user; and
the step of generating a distribution discrepancy report is followed by the step of generating an agency report by the reporting module for one of the agency users analyzing the inventory records and vaccination records associated with the vaccination sites encompassed within the jurisdiction of the agency user.

19. The method as recited in claim 18, wherein:
the step of creating a vaccination record further comprises generating a timestamp in real-time stored within the vaccination record by the vaccination verification module, and the vaccination verification module preventing any of the users from modifying the timestamp.

20. The method as recited in claim 19, wherein:
the step of referencing the vaccination record associated with the patient identifier is followed by the steps of:
submitting side effect data associated with one of the patients to the reporting module by one of the medical users or the patient user via the user devices, storing the side effect data within the vaccination record associated with said patient, and identifying the lot and manufacturer of the vaccine dose administered to the patient via the lot identifier of the vaccination record.
